(19) United States
(12) Patent Application Publication
(10) Pub. No.: US 2012/0301410 A1
(43) Pub. Date: Nov. 29, 2012

(54) COSMETIC

(76) Inventor: Shahena ALI, Stanford-Le-Hope (GB)

(21) Appl. No.: 13/410,248

(22) Filed: Mar. 1, 2012

(30) Foreign Application Priority Data
Mar. 1, 2011 (GB) 1103498.0

(51) Int. Cl. A61K 8/97; A61Q 1/04; A61Q 17/02

(52) U.S. Cl. 424/59

(57) ABSTRACT

A cosmetic for application to skin, the cosmetic comprising a base matrix to coat a skin surface as a wet covering to present a pheromone component within for contact transfer release with or without the base matrix.

### BACKGROUND OF THE INVENTION

The present invention relates generally to cosmetics and more particularly to lip cosmetics such as gloss, balm and lipsticks but also may be used with cosmetic in general such as a colour cosmetic or beauty product, a make up, a non-colour cosmetic or beauty product, a skincare product, a body care product, a hair care product or fragrance product.

It is well known that to improve self-esteem and appearance many individuals and in particular women wear lip cosmetics in the form of lipsticks and glosses. Such cosmetics come in a variety of colours, sheens and application methods as wells as latterly may have pleasant flavours. It will be appreciated that some cosmetics such as lip cosmetics are tasted during intimate acts such as kissing but of course intimate contact can also occur on other parts of the body. Thus, in the example the nature of the lip cosmetic colour as well as shimmer can all be considered part of the self-perceived improvement in appearance but also how an individual is perceived particularly by members of the opposite sex. Lip cosmetics are part of an initial first sight between a couple and also the next stages of physical intimacy.

### SUMMARY OF THE INVENTION

In accordance with aspects of the present invention there is provided a cosmetic, the cosmetic comprising a base matrix to coat a skin surface as a wet covering to present a pheromone component within for contact transfer release with or without the base matrix.

The base matrix may be formulated to facilitate contact transfer release by compression and/or heat and/or wetting. The base matrix may be formulated to facilitate contact transfer release within a desired time period and/or for a desired time period and/or at a desired contact transfer release rate. The base matrix may be formulated to form a surface skin after application in use to inhibit release of the pheromone component prior to contact transfer release. The cosmetic may have a second application coating to provide the surface skin.

The base matrix may be a lip gloss.

The cosmetic may be a lip cosmetic or a colour/beauty product or make-up or non-colour cosmetic/beauty product or skincare product or body care product or hair care product or fragrance.

The pheromone component may be Oxytocin and/or Copulin and/or Estratetraenol. The pheromone component may comprise a pheromone complex comprising more than one pheromone. The pheromone complex may be (Oxytocin and Copulin) or (Oxytocin and Estratetraenol) or (Copulin and Estratetraenol) or (Oxytocin and Copulin and Estratetraenol).

In a first aspect of the present invention the base matrix may contain the following components:

Polybutene,

ParaffinumLiquidum

OctylPalmitate

Ethylene/Propylene/Styrene Copolymer

Butylene/Ethylene/Styrene Copolymer

Octyldodecanol

DisteardimoniumHectorite

Flavour

Propylene Carbonate

Tocopheryl Acetate (Vitamin E)

BHT

Maxilip™

Matrixyl 3000™ (Palmitoyl Pentapeptide-3)

Hawaiian Kukui Nut Oil (Desert Jojoba) (Vitamin A, E, and F)

Collagen

Anti-UV stress (Eusolex 2292)(Sunscreen)

Shea Butter

(Copulin) Pheromones (Proprietary Formula)

Niacinamide (Vitamin B3)

Ceramide Lipid (Cerasol™)

Hyaluronic Acid

L-Arginine

In accordance with second aspects of the present invention the base matrix may contain:

Polybutene,

ParaffinumLiquidum

OctylPalmitate

Ethylene/Propylene/Styrene Copolymer

Butylene/Ethylene/Styrene Copolymer

Octyldodecanol

DisteardimoniumHectorite

Flavour

Propylene Carbonate

Tocopheryl Acetate (Vitamin E)

BHT

Maxilip™

Matrixyl 3000™ (Palmitoyl Pentapeptide-3)

Hawaiian Kukui Nut Oil (Desert Jojoba) (Vitamin A, E, and F)

Collagen

Anti-UV stress (Eusolex 2292)(Sunscreen)

Shea Butter

(Oxytocin) Pheromones (Proprietary Formula)

Niacinamide (Vitamin B3)

Ceramide Lipid (Cerasol™)

Hyaluronic Acid

L-Arginine

In accordance with third aspects of the present invention the base matrix may include:

Polybutene,

ParaffinumLiquidum

OctylPalmitate

Ethylene/Propylene/Styrene Copolymer

Butylene/Ethylene/Styrene Copolymer

Octyldodecanol

DisteardimoniumHectorite

Flavour

Propylene Carbonate

Tocopheryl Acetate (Vitamin E)

BHT

Maxilip™

Matrixyl 3000™ (Palmitoyl Pentapeptide-3)

Hawaiian Kukui Nut Oil (Desert Jojoba) (Vitamin A, E, and F)

Collagen

Anti-UV stress (Eusolex 2292)(Sunscreen)

Shea Butter

(Estratetraenol) Pheromones (Proprietary Formula)

Niacinamide (Vitamin B3)

Ceramide Lipid (Cerasol™)

Hyaluronic Acid

L-Arginine

Aspects of the present may include in the base matrix Calcium Aluminium Borosilicate.

### DETAILED DESCRIPTION

Embodiments of the invention will now be described by way of example only.

A pheromone is normally secreted or excreted chemical factor that triggers a social response in members of the same species. Pheromones are chemicals capable of acting outside of the body of the secreting individual to impact the behaviour of the receiving individual. Natural pheromones are collected and synthetic pheromones manufactured to provide particular responses and in particular sexual responses either positive or negative. Thus, a male or a female may wish to attract or maintain the attraction of a member of the opposite sex or may wish to deter attention by use of pheromones. It will be appreciated that a disadvantage of pheromones is that the pheromone may diffuse away faster than it can be provided and it may take too long to accumulate to an effective concentration.

With humans it will be understood that social convention now deters general responses between all females and all males with generally now an accepted process being a first visual attractiveness and a subsequent more direct acquaintance. Unfortunately, in crowded places such as night clubs and bars there may be a large number of individuals each producing pheromones so there can be confusion as to social triggers. Aspects of the present invention aim to address such problems by providing a cosmetic base matrix which has a self-esteem enhancing function through expected colour, look and texture but also provides a base to present a pheromone component for contact transfer release, as well as nascent vapour release if desired, and so focussed presentation to a recipient after an initial visual attraction. One or more of the following pheromones may be provided in the cosmetic; Oxytocin, Copulin and/or Estratetraenol.

The example particularly given below is as a cosmetic lip gloss so a user in such circumstances not only chooses a pheromone which he or she likes but also by implication will be attractive to biologically, if not emotionally compatible recipients, in combination with the recipients own pheromones; individuals who like the same pheromones will generally be attracted to each other. The lip gloss with a pheromone component in such circumstances reinforces the situation of interaction in particularly crowded situations where relatively large numbers of individuals will be in close proximity to each other. It will also be understood in a converse situation a user may use pheromones to deter attention or for example if an individual wishes to break up with a recipient use a different pheromone to make themselves less attractive to the recipient. Although as indicated above the particular embodiments described are lip cosmetics/gloss so the base matrix is a lip gloss formulation it will be understood that other cosmetics can be formulated. In such circumstances it will be appreciate that the cosmetic may be provide in more intimate parts of the body when individuals have developed their relationship to that extent or a combination of a highly attractive pheromone provide as a lip gloss and a discouraging pheromone in other cosmetics such as a body lotions upon more intimate parts of the body.

The base matrix has many functions as indicated above. The matrix may be transparent and substantially colourless but more normally will have typical lip gloss colours so that the cosmetic will not be readily distinguished from normal lip gloss and cosmetics. Thus, the matrix will generally lie as a covering over a user's lips with a wet or at least substantially moist tactile nature such that in contact with a recipient there is transfer of the matrix or the pheromone to the recipient. In accordance with aspects of the present invention this contact transfer release is facilitated by formulation of the base matrix of the cosmetic.

It will be appreciated that base matrixes will normally be formed from natural fat or oil or water base components in order to thicken as perceived by an observer the lips of a user. A relatively thick layer of base matrix will be applied. During a kiss the lips of the user will engage the lips of a potential recipient with a degree of contact force whereby the cosmetic as a lip gloss will be compressed and possibly smear so that such action may release or cause transfer of pheromones to the recipient.

It will be appreciated during a kiss there may be sustained contact between a user of the lip cosmetic and a potential recipient. Due to the nature of the contact and possibly enhanced blood flow heat may be generated which can enhance pheromone release from the matrix to the recipient.

It will be appreciated that during kissing there will typically be a degree of saliva exchange as a fluid in addition to actual drinks consumed by the user and/or the potential recipient which may again facilitate pheromone release. Furthermore, alcohol in such drinks may act to facilitate or block release of pheromone as required.

All of the above means to facilitate pheromone release can act independently or in concert to provide desired pheromone release. It will be understood that the cosmetic will be designed to provide and achieve a certain level of pheromone release within a desired time period, the rate of release and the period of time for such release. It may even be possible to switch to different pheromones or mixes of pheromones or transferred concentrations of pheromones over a period to avoid de-sensitising the user and/or the recipient. Such means may be through different sized micro-encapsulation of the pheromone or particle sizes of the pheromone or other techniques understood by skilled formulators and persons skilled in the technology.

It will be appreciated that pheromones, whether natural or semi-synthetic or synthetic, are relatively expensive. Thus, judicious use is important and release without effect a waste. Thus, the lip gloss base matrix in accordance with aspects of the present invention may be formulated to form a thin skin which inhibits high level pheromone release either as vapour into the air on a user's breath unless desired or onto a drinks receptacle again unless desired for example to begin a period or another period of airborne release of pheromone in the vicinity of a potential recipient. A further approach may be to provide a second application barrier or coating as the skin and possibly in the form of a spray to either inhibit pheromone release until desired or ‘top up’ the pheromone level in the base matrix.

A first embodiment of a cosmetic in accordance with aspects of the present invention has a formulation including:

Polybutene,

ParaffinumLiquidum

OctylPalmitate

Ethylene/Propylene/Styrene Copolymer

Butylene/Ethylene/Styrene Copolymer

Octyldodecanol

DisteardimoniumHectorite

Flavour

Propylene Carbonate

Tocopheryl Acetate (Vitamin E)

BHT

Maxilip™

Matrixyl 3000™ (Palmitoyl Pentapeptide-3)

Hawaiian Kukui Nut Oil (Desert Jojoba) (Vitamin A, E, and F)

Collagen

Anti-UV stress (Eusolex 2292)(Sunscreen)

Shea Butter

(Copulin) Pheromones (Proprietary Formula)

Niacinamide (Vitamin B3)

Ceramide Lipid (Cerasol™)

Hyaluronic Acid

L-Arginine

The first embodiment has the following effects to combine the self-esteem aspects of a cosmetic with the pheromone release aspects of the present invention:

Contains Copulin Pheromones to have the men falling at your feet!

Improve the lip contour with Maxi Lip™ (clinical studies show up to 40% increase in lip volume, 30% reduction in surface folds and 60% improvement in hydration in 29 days).

Enriched with a natural plumping agent and sensual cooling sensation to give a fuller shape to lips with long-lasting, richly pigmented colour.

Anti-Ageing with effective UV protection (Sunscreen) to prevent wrinkles and sun-damage and Pentapeptides (Matrixyl™) to increase lip volume by stimulating Collagen and Elastin production.

Shea Butter and Collagen nourish and moisturize lips for a youthful appearance.

Hyaluronic Acid molecules swell to up to 30 times their own size working directly with the body's hydration system to collect moisture, reduce wrinkles and plump lips.

Extreme lip-plumping action with L-Arginine amino acid to stimulate Collagen production and blood flow to the skin surface for fuller lips.

Niacinamide (Vitamin B3) plumps lips by increasing circulation to the lip area, creating a natural rosy colour to the lips by dilating the blood vessels.

Repair and regenerate lips with Ceramide Lipids (Cerasol™) to compensate for a decrease of ceramides in aging skin.

Moisturizing with Hawaiian Kukui Nut Oil (Desert Jojoba), a super emollient with Vitamin A, E, and F, offering free radical antioxidant protection.

A second embodiment of aspects of the present invention has a formulation including:

Polybutene,

ParaffinumLiquidum

OctylPalmitate

Ethylene/Propylene/Styrene Copolymer

Butylene/Ethylene/Styrene Copolymer

Octyldodecanol

DisteardimoniumHectorite

Flavour

Propylene Carbonate

Tocopheryl Acetate (Vitamin E)

BHT

Maxilip™

Matrixyl 3000™ (Palmitoyl Pentapeptide-3)

Hawaiian Kukui Nut Oil (Desert Jojoba) (Vitamin A, E, and F)

Collagen

Anti-UV stress (Eusolex 2292)(Sunscreen)

Shea Butter

(Oxytocin) Pheromones (Proprietary Formula)

Niacinamide (Vitamin B3)

Ceramide Lipid (Cerasol™)

Hyaluronic Acid

L-Arginine

The second embodiment has the following effects to combine the self-esteem aspects of a cosmetic with the pheromone release aspects of the present invention:

Contains Oxytocin Pheromones to make the men fall in love with you!

Improve the lip contour with Maxi Lip™ (clinical studies show up to 40% increase in lip volume, 30% reduction in surface folds and 60% improvement in hydration in 29 days).

Enriched with a natural plumping agent and sensual cooling sensation to give a fuller shape to lips with long-lasting, richly pigmented colour.

Anti-Ageing with effective UV protection (Sunscreen) to prevent wrinkles and sun-damage and Pentapeptides (Matrixyl™) to increase lip volume by stimulating Collagen and Elastin production.

Shea Butter and Collagen nourish and moisturize lips for a youthful appearance.

Hyaluronic Acid molecules swell to up to 30 times their own size working directly with the body's hydration system to collect moisture, reduce wrinkles and plump lips.

Extreme lip-plumping action with L-Arginineamino acid to stimulate Collagen production and blood flow to the skin surface for fuller lips.

Niacinamide (Vitamin B3) plumps lips by increasing circulation to the lip area, creating a natural rosy colour to the lips by dilating the blood vessels.

Repair and regenerate lips with Ceramide Lipids (Cerasol™) to compensate for a decrease of ceramides in ageing skin.

Moisturize with Hawaiian Kukui Nut Oil (Desert Jojoba); a super emollient with Vitamin A, E, and F, offering free radical antioxidant protection.

A third embodiment of aspects of the present invention has a formulation including:

Polybutene,

ParaffinumLiquidum

OctylPalmitate

Ethylene/Propylene/Styrene Copolymer

Butylene/Ethylene/Styrene Copolymer

Octyldodecanol

DisteardimoniumHectorite

Flavour

Propylene Carbonate

Tocopheryl Acetate (Vitamin E)

BHT

Maxilip™

Matrixyl 3000™ (Palmitoyl Pentapeptide-3)

Hawaiian Kukui Nut Oil (Desert Jojoba) (Vitamin A, E, and F)

Collagen

Anti-UV stress (Eusolex 2292)(Sunscreen)

Shea Butter

(Estratetraenol) Pheromones (Proprietary Formula)

Niacinamide (vitamin B3)

Ceramide Lipid (Cerasol™)

Hyaluronic Acid

L-Arginine

The third embodiment has the following effects to combine the self-esteem aspects of a cosmetic with the pheromone release aspects of the present invention:

Contains Estratetraenol Pheromones to make you totally irresistible!

Improve the lip contour with Maxi Lip™ (clinical studies show up to 40% increase in lip volume, 30% reduction in surface folds and 60% improvement in hydration in 29 days).

Enriched with a natural plumping agent and sensual cooling sensation to give a fuller shape to lips with long-lasting, richly pigmented colour.

Anti-Ageing with effective UV protection (Sunscreen) to prevent wrinkles and sun-damage and Pentapeptides (Matrixyl™) to increase lip volume by stimulating Collagen and Elastin production.

Shea Butter and Collagen nourish and moisturize lips for a youthful appearance.

Hyaluronic Acid molecules swell to up to 30 times their own size working directly with the body's hydration system to collect moisture, reduce wrinkles and plump lips.

Extreme lip-plumping action with L-Arginine amino acid to stimulate Collagen production and blood flow to the skin surface for fuller lips.

Niacinamide (Vitamin B3) plumps lips by increasing circulation to the lip area, creating a natural rosy colour to the lips by dilating the blood vessels.

Repair and regenerate lips with Ceramide Lipids (Cerasol™) to compensate for a decrease of ceramides in aging skin.

Moisturizing with Hawaiian Kukui Nut Oil (Desert Jojoba), a super emollient with Vitamin A, E, and F, offering free radical antioxidant protection

Embodiments of aspects of the present invention may also include Calcium Aluminium Borosilicate.

As indicated above pheromones may be mixed in a pheromone complex for enhances effects. Thus, a pheromone component in the cosmetic may be Oxytocin or Copulin or Estratetraenol substantially alone or combined such as (Oxytocin and Copulin) or (Oxytocin and Estratetraenol) or (Copulin and Estratetraenol) or (Oxytocin and Copulin and Estratetraenol) together in desired proportions by weight or volume.

Modifications and alterations in accordance with aspects of the present invention will be understood by persons skilled in the technology so described as a cosmetic it will also be appreciated the cosmetic may be edible as a confectionary or chocolate in which the base matrix is appropriately formulated. The colour or shade of the cosmetic may be associated with the concentration or rate of contact release of pheromone whereby the correct cosmetic can be chosen for a particular environment or encounter. The colour of the cosmetic may change with age or exposure to air so that a user may be aware of the diminishing strength of the pheromone in the cosmetic.

It will be appreciated by those skilled in the art that any number of combinations of the aforementioned features and/or those shown in the appended drawings provide clear advantages over the prior art and are therefore within the scope of the invention described herein.

1. A cosmetic for application to skin, the cosmetic comprising a base matrix to coat a skin surface as a wet covering to present a pheromone component within for contact transfer release with or without the base matrix.

2. A cosmetic as claimed in claim 1 wherein the base matrix is formulated to facilitate contact transfer release by compression and/or heat and/or wetting.

3. A cosmetic as claimed in claim 1 wherein the base matrix is formulated to facilitate contact transfer release within a desired time period and/or for a desired time period and/or at a desired contact transfer release rate.

4. A cosmetic as claimed in claim 1 wherein the base matrix is formulated to form a surface skin after application in use to inhibit release of the pheromone component prior to contact transfer release.

5. A cosmetic as claimed in claim 4 wherein the cosmetic has a second application coating to provide the surface skin.

6. A cosmetic as claimed in claim 1 wherein the cosmetic is a lip cosmetic or a colour/beauty product or make-up or non-colour cosmetic/beauty product or skincare product or body care product or hair care product or fragrance

7. A cosmetic as claimed in claim 1 wherein the base matrix is a lip gloss formulation.

8. A cosmetic as claimed in claim 1 wherein the pheromone component may be Oxytocin and/or Copulin and/or Estratetraenol.

9. A cosmetic as claimed in claim 1 wherein the pheromone component comprises a pheromone complex comprising more than one pheromone.

10. A cosmetic as claimed in claim 9 wherein the pheromone complex is (Oxytocin and Copulin) or (Oxytocin and Estratetraenol) or (Copulin and Estratetraenol) or (Oxytocin and Copulin and Estratetraenol) in desired relative proportions by weight or volume.

11. A cosmetic as claimed in claim 1 wherein the base matrix contains a combination of one or more of the following components:
Polybutene,
ParaffinumLiquidum
OctylPalmitate
Ethylene/Propylene/Styrene Copolymer
Butylene/Ethylene/Styrene Copolymer
Octyldodecanol
DisteardimoniumHectorite
Flavour
Propylene Carbonate
Tocopheryl Acetate (Vitamin E)
BHT
Maxilip™
Matrixyl 3000™ (Palmitoyl Pentapeptide-3)
Hawaiian Kukui Nut Oil (Desert Jojoba) (Vitamin A, E, and F)
Collagen
Anti-UV stress (Eusolex 2292)(Sunscreen)
Shea Butter
(Copulin) Pheromones (Proprietary Formula)
Niacinamide (Vitamin B3)
Ceramide Lipid (Cerasol™)
Hyaluronic Acid
L-Arginine.

12. A cosmetic as claimed in claim 1 wherein the base matrix contains one or more of the following components:
Polybutene,
ParaffinumLiquidum
OctylPalmitate
Ethylene/Propylene/Styrene Copolymer
Butylene/Ethylene/Styrene Copolymer
Octyldodecanol
DisteardimoniumHectorite
Flavour
Propylene Carbonate
Tocopheryl Acetate (Vitamin E)
BHT
Maxilip™
Matrixyl 3000™ (Palmitoyl Pentapeptide-3)
Hawaiian Kukui Nut Oil (Desert Jojoba) (Vitamin A, E, and F)
Collagen
Anti-UV stress (Eusolex 2292)(Sunscreen)
Shea Butter
(Oxytocin) Pheromones (Proprietary Formula)
Niacinamide (Vitamin B3)
Ceramide Lipid (Cerasol™)
Hyaluronic Acid
L-Arginine

13. A cosmetic as claimed in claim 1 wherein the base matrix includes one or more of the following components:
Polybutene,
ParaffinumLiquidum
OctylPalmitate
Ethylene/Propylene/Styrene Copolymer
Butylene/Ethylene/Styrene Copolymer
Octyldodecanol
DisteardimoniumHectorite
Flavour
Propylene Carbonate
Tocopheryl Acetate (Vitamin E)
BHT
Maxilip™
Matrixyl 3000™ (Palmitoyl Pentapeptide-3)
Hawaiian Kukui Nut Oil (Desert Jojoba) (Vitamin A, E, and F)
Collagen
Anti-UV stress (Eusolex 2292)(Sunscreen)
Shea Butter
(Estratetraenol) Pheromones (Proprietary Formula)
Niacinamide (Vitamin B3)
Ceramide Lipid (Cerasol™)
Hyaluronic Acid
L-Arginine

14. A cosmetic as claimed in claim 1 wherein the base matrix includes Calcium Aluminium Borosilicate.
